(19) United States
(12) Patent Application Publication
(10) Pub. No.: US 2008/0146864 A1
(43) Pub. Date: Jun. 19, 2008

(54) MAGNETIC THERAPEUTIC DEVICE AND METHOD FOR PRODUCING MAGNETIC THERAPEUTIC DEVICE

(75) Inventors: Yoshinobu Arai, Osaka-shi (JP); Kouei Irisawa, Numata-shi (JP); Misako Irisawa, Numata-shi (JP)

Correspondence Address: CASELLA & HESPOS, 274 MADISON AVENUE, NEW YORK, NY, 10016, US

(73) Assignee: PIP-FUJIMOTO CO., LTD., Osaka-shi (JP)

(21) Appl. No.: 11/954,768

(22) Filed: Dec. 12, 2007

(30) Foreign Application Priority Data
Dec. 18, 2006 (JP) 2006-339986
Jan. 25, 2007 (JP) 2007-014472

(51) Int. Cl. A61N 2/08; B21D 39/00

(52) U.S. Cl. 600/15; 29/428

(57) ABSTRACT

Disclosed is a magnetic therapeutic device for use as a wearable article including a necklace to exert a blood flow-promoting effect on a body of a user. The magnetic therapeutic device comprises an elongated flexible magnet portion formed from a mixture of a silicone rubber and a magnetic powder to extend in a given direction, and magnetically polarized, and a flexible sheath portion formed from a silicone rubber to cover over an entire outer periphery of the magnet portion. The magnetic therapeutic device of the present invention can more uniformly apply a magnetic flux to a body of a user.

### BACKGROUND OF THE INVENTION

1. Field of the Invention

The present invention relates to a magnetic therapeutic device having a blood flow-promoting effect and others, and a method for producing a magnetic therapeutic device.

2. Description of the Related Art

Heretofore, it has been noted that a magnetic flux applied to a human body can promote blood flow (i.e., blood circulation) and provide favorable health effects, and therefore various health promoting devices have been developed to obtain such advantages.

For example, Japanese Unexamined Patent Publication No. 11-111515 discloses an elongated magnetic member for use as a wearable article, such as a magnetic necklace, wherein a magnetic powder emitting a magnetic flux is mixed therein. This magnetic member comprises a magnet portion formed by a process of mixing a ferrite powder with a synthetic resin material in an amount of 80 weight % or more, heating and softening the mixture, and shaping the softened mixture into a cord shape through the use of an extruder, and a sheath portion formed by a process of heating and softening a thermoplastic elastomer and extruding the softened thermoplastic elastomer around an outer periphery of the magnet portion through the use of an extruder.

This magnetic member makes it possible to achieve a magnetic necklace capable of preventing a material of the magnet portion, such as a ferrite powder, from becoming exposed to outside and coming into direct contact with a skin of a user, while maintaining a given magnetic intensity.

In the above magnetic member, each of the magnet portion and the sheath portion is formed from a different material. Consequently, a contact between the magnet portion and the sheath portion is liable to become insufficient or uneven, which is likely to cause difficulty in managing a magnetic intensity to be emitted outside from the magnet portion, and preclude the capability of uniformly applying a magnetic flux from the magnet portion to a body of a user.

### SUMMARY OF THE INVENTION

In view of the above circumstances, it is an object of the present invention to provide a magnetic therapeutic device which can more uniformly apply a magnetic flux to a body of a user, and a method for producing a magnetic therapeutic device.

According to an aspect of the present invention, a magnetic therapeutic device is adapted to exert a blood flow-promoting effect on a body of a user. The magnetic therapeutic device comprises an elongated flexible magnet portion formed from a mixture of a silicone rubber and a magnetic powder to extend in a given direction, and magnetically polarized, and a flexible sheath portion formed from a silicone rubber to cover over an entire outer periphery of the magnet portion.

These and other objects, features and advantages of the invention will become more apparent upon reading the following detailed description along with the accompanying drawings.

### BRIEF DESCRIPTION OF THE DRAWINGS

FIG. 1 is a front view showing a magnetic necklace serving as a magnetic therapeutic device according to an embodiment of the present invention.

FIG. 2 is a sectional view showing the magnetic necklace illustrated in FIG. 1.

FIG. 3 is a schematic front view showing an extrusion molding apparatus for use in producing the magnetic necklace illustrated in FIG. 1.

FIG. 4 is a schematic top view showing the extrusion molding apparatus illustrated in FIG. 3.

FIG. 5 is a schematic sectional view showing a die for use in the extrusion molding apparatus illustrated in FIG. 3.

FIG. 6 is a schematic sectional view showing a mold assembly for use in providing an engagement portion to the magnetic necklace illustrated in FIG. 1.

FIG. 7 is a schematic sectional view showing another example of the die for use in the extrusion molding apparatus illustrated in FIG. 3.

FIG. 8 is a fragmentary side view showing one example of modification of the magnetic necklace illustrated in FIG. 1.

### DETAILED DESCRIPTION OF THE PREFERRED EMBODIMENTS

With reference to the drawings, the present invention will now be described based on an preferred embodiment thereof. The following description will be made about one example where a magnetic therapeutic device of the present invention is used as a magnetic necklace.

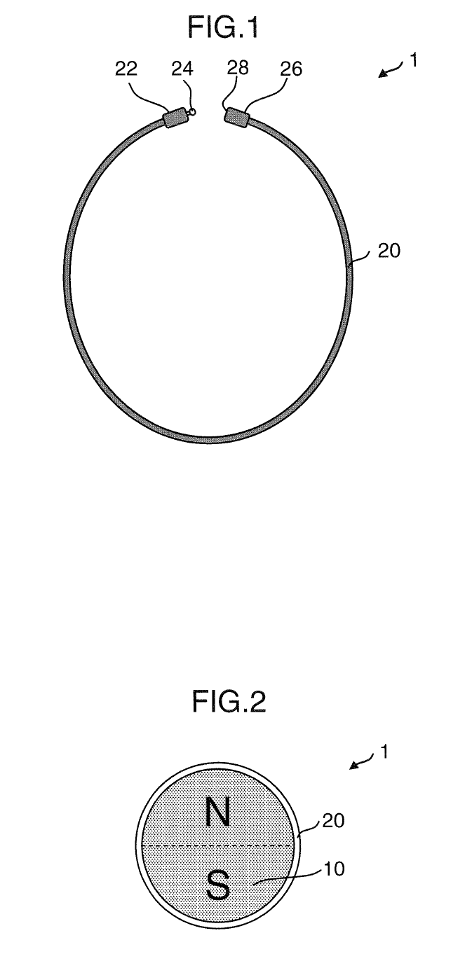

FIG. 1 is a front view showing a magnetic necklace which is a magnetic therapeutic device according to one embodiment of the present invention, and FIG. 2 is a sectional view showing the magnetic necklace.

As shown in FIGS. 1 and 2, the magnetic necklace 1 comprises a magnet portion 10, a sheath portion 20, a lock portion 22 and an engagement portion 26.

The magnet portion 10 is provided as a means to apply a magnetic flux to a body of a user. In this embodiment, this magnet portion 10 is formed as an elongated member having a diameter of about 3 mm. The magnet portion 10 is formed by subjecting a mixture of a silicone rubber and a magnetic powder (i.e., silicone rubber-and-magnetic powder mixture) to an extrusion molding process, to have flexibility in its entirety based on resilience of the silicone rubber. In this embodiment, the magnetic powder is a mixed powder consisting of a powder of ferrite magnet and a powder of rear-earth magnet having a relatively high magnetic flux intensity. A mixing ratio of the magnetic powder to the silicone rubber for the magnet portion 10 is set at about 7:3. The magnet portion 10 is magnetically polarized. Specifically, as shown in FIG. 2, the magnet portion 10 is polarized in such a manner that two magnetic poles are formed on respective opposite outer sides thereof.

The sheath portion 20 is provided as a means to cover over an entire outer periphery of the magnet portion 10 so as to prevent the magnet powder contained in the magnet portion 10 from escaping or spilling outside. In this embodiment, the sheath portion 20 is formed as a member having a thickness of about 0.3 mm. The sheath portion 20 is formed by subjecting a silicone rubber to an extrusion molding process. The sheath portion 20 has flexibility enough to readily deform as shown in FIG. 1 while enclosing the magnet portion 10 therein.

A silicone rubber is considered as a material having higher physiological stability and less adverse effects on a human body as compared with other synthetic resins. Thus, the sheath portion 20 formed from the silicone rubber to cover over the magnet portion 10 makes it possible to more reliably suppress an adverse effect on a skin of a user when the magnetic necklace 1 is brought into direct contact with the user's skin, while preventing the magnetic powder from attaching onto the user's skin.

The lock portion 22 is provided as a means to lock the engagement portion 26. In this embodiment, the lock portion 22 is formed from a silicone rubber at one of opposite ends of the sheath portion 20. The lock portion 22 is provided with a lock protrusion 24 protruding outwardly from the sheath portion 20. The lock protrusion 24 is adapted to come into engagement with an after-mentioned engagement hole 28 of the engagement portion 26 so as to lock the engagement portion 26.

The engagement portion 26 is adapted to be locked by the lock portion 22. Specifically, the engagement portion 26 is provided at the other end of the sheath portion 20 on an opposite side of the lock portion 22. The engagement portion 26 is formed with an engagement hole 28 engageable with the lock protrusion 24 in a releasable manner. The magnetic necklace 1 is adapted, when the engagement hole 28 and the lock protrusion 24 are brought into engagement with each other, to have a ring shape.

When the above magnetic necklace 1 is attached, i.e., worn, around a neck of a user through the engagement between the lock protrusion 24 and the engagement hole 28, the sheath portion 20 is bent and brought into direct contact with a skin around the neck, while maintaining the condition where it covers over the magnet portion 10. Thus, a magnetic flux from the magnet portion 10 will be applied to the user's body to exert a magnetic effect, such as a blood flow-promoting effect on the user's body. In the magnetic necklace 1, a magnetic intensity from the magnet portion 10 is at about 50 mT.

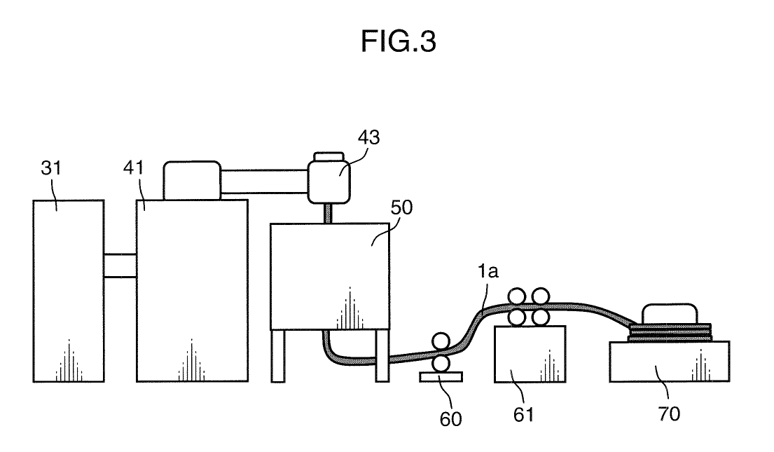

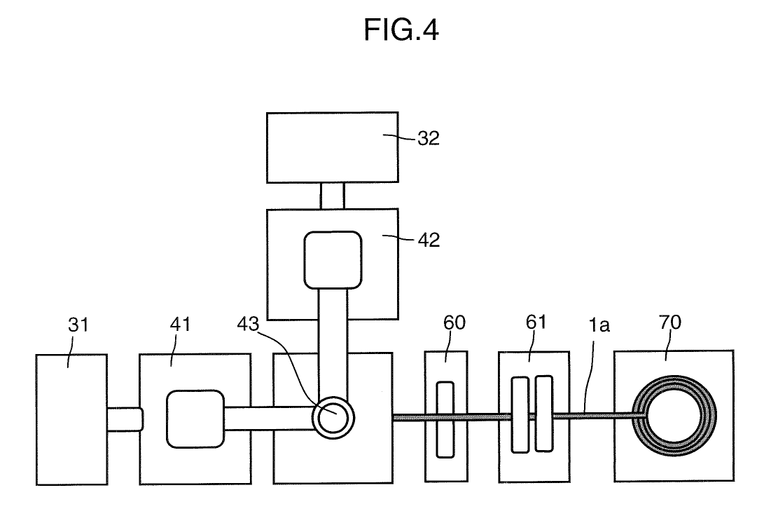

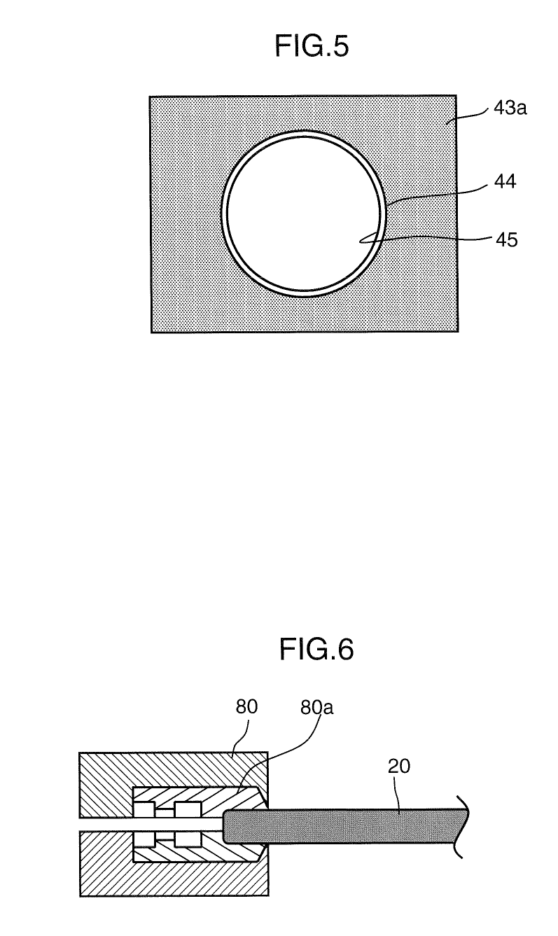

A production method for the above magnetic necklace 1 will be described below with reference to the drawings. FIG. 3 is a schematic front view showing an extrusion molding apparatus for use in extrusion-molding the magnetic necklace, and FIG. 4 is a schematic top view showing the extrusion molding apparatus in FIG. 3. FIG. 5 is a schematic sectional view showing a die for use in the extrusion molding apparatus. The extrusion molding apparatus comprises a first kneader 31, a second kneader 32, a first extruder 41, a second extruder 42, a vulcanizer 50, two pullers 60, 61 and a winder 70.

The production method for the magnetic necklace 1 using the extrusion apparatus includes the following steps.

(1) Mastication Step

A mastication step is performed to masticate and plasticize a millable-type silicone rubber for each of the magnet portion 10 and the sheath portion 20. In this mastication step, a crude rubber of the millable-type silicone rubber is repeatedly kneaded by a roller in each of the first and second kneaders 31, 32, and gradually plasticized.

(2) Kneading Step

A kneading step is performed to kneading the plasticized millable-type silicone rubber together with an additional material. In the kneading step, the millable-type silicone rubber, the rear-earth magnet powder, the ferrite magnet powder, and a vulcanizing agent, such as an organic peroxide, are put into the first kneader 31, and kneaded and homogeneously mixed together to obtain a magnet-side mixture. Further, the millable-type silicone rubber, and a vulcanizing agent, such as an organic peroxide, are put into the second kneader 32, and kneaded and homogeneously mixed together to obtain a sheath-side mixture.

(3) Charging Step

A charging step is performed to charge each of the magnet-side and sheath-side mixtures kneaded in the kneading step, into a corresponding one of the first and second extruders 41, 42. Specifically, in the charging step, the magnet-side mixture kneaded in the first kneader 31 is charged into the first extruder 41, and the sheath-side mixture kneaded in the second kneader 32 is charged into the second extruder 42.

(4) Extrusion Step

An extrusion step is performed to extrude each of the charged magnet-side and sheath-side mixtures from the corresponding one of the first and second extruders 41, 42. Each of the first and second extruders 41, 42 is connected to a combining unit 43 disposed above the vulcanizer 50. Specifically, the combining unit 43 is provided with a die 43a as shown in FIG. 5. The die 43a comprises a cylindrical-shaped inner frame 45 formed of a thin-walled plate member to extending vertically, and an outer frame 44 enclosing the inner frame 45. An outlet port of the first extruder 41 is coupled to an inlet port of the inner frame 45, and an outlet port of the second extruder 42 is coupled to an inlet port of an annular-shaped space surrounded by the inner and outer frames 45, 44.

In the extrusion step, the first extruder 41 operates to extrude the magnet-side mixture charged in the first extruder 41, from an outlet port of the inner frame 45 downwardly through an internal space thereof. Simultaneously, the second extruder 42 operates to the sheath-side mixture charged in the second extruder 42, from an outlet port of the annular-shaped space surrounded by the inner and outer frames 45, 44 downwardly. In this manner, the sheath-side mixture is extruded in such a manner as to cover over the extruded magnet-side mixture, and the combined magnet-side and sheath-side mixtures are continuously fed toward the vulcanizer 50.

(5) Vulcanization Step

A vulcanization step is performed to vulcanize the combined magnet-side and sheath-side mixtures extruded from the first and second extruders 41, 42. In the vulcanization step, hot air at an ambient pressure and about 200° C. is supplied to an outer periphery of the sheath-side mixture covering over the magnet-side mixture, to vulcanize and cure the millable-type silicone rubber contained in each of the magnet-side and sheath-side mixtures. During this step, the silicone rubber in the magnet-side mixture and the silicone rubber in the sheath-side mixture are vulcanized under a condition that they are in contact with each other. Thus, the respective silicone rubbers are cured to allow the magnet portion 10 and the sheath portion 20 to be integrated together. In this manner, through the vulcanization step, a cord-shaped magnetic member 1a having an integral structure of the magnet portion 10 and the sheath portion 20 covering over the magnet portion 10 is formed.

(6) Winding Step

A winding step is performed to wind the cord-shaped magnetic member 1a discharged from the vulcanizer 50 in the form of an integral structure of the magnet portion 10 and the sheath portion 20. In the winding step, the magnetic member 1a is pulled by the pullers 60, 61 each having a roller unit, and sequentially wound by the winder 70.

(7) Cutting Step

A cutting step is performed to cut the cord-shaped magnetic member 1a wound by the winder 70, to a given length.

(8) Lock-Section Fusion-Bonding Step

A lock-section fusion-bonding step is performed to fusion-bond a lock section consisting of the lock portion 22 and the engagement portion 26, to the sheath portion 20 of the magnetic member 1a cut to the given length. In the lock-section fusion-bonding step, a first one of opposite ends of the sheath portion 20 is attached to an engagement portion-side mold assembly 80 including a pattern 80a for the lock portion 22 as shown in FIG. 6. Then, a millable-type silicone rubber sheet formed separately is inserted into the mold assembly 80. Subsequently, the sheet is fusion-bonded to the first end of the sheath portion 20 while applying a given molding pressure to the mold assembly 80. In the same manner, the lock portion 22 is formed at the other, i.e., second, end of the sheath portion 20. Through the lock-section fusion-bonding step, an outer shape of the magnetic necklace 1 is formed.

(9) Polarization Step

In a polarization step, a high magnetic field is applied to the magnet portion 10 covered by the sheath portion 20 to magnetically polarize the magnet portion 10.

Through the polarization step, the magnet portion 10 is formed as a magnet having N- and S-poles each arranged along a longitudinal direction there. In the above manner, the magnetic necklace 1 capable of uniformly applying a magnet flux of the magnet portion 10 to a body of a user is formed.

As above, in the magnetic necklace 1 according to the above embodiment, the magnet portion 10 formed from the silicone rubber mixed with the magnetic powder is covered by the sheath portion 20 formed from the silicone rubber. This makes it possible to prevent the magnetic powder from attaching to a skin of a user or the like and damaging the user's skin. In addition, the magnet portion 10 is formed from the silicone rubber-and-magnetic powder mixture, and the sheath portion 20 is formed from the same silicone rubber as that of the magnet portion 10. This allows the magnet portion 10 and the sheath portion 20 to be in contact with each other with sufficient conformability, so as to facilitate uniformization of a magnetic flux to be emitted outside from the magnet portion 10, and effectively exert a blood flow-promoting effect of the magnetic flux on a body of the user. Furthermore, the silicone rubber is a material having physiological stability, and therefore an adverse effect of the sheath portion on the skin can be more reliably suppressed.

In the above embodiment, a powder of rear-earth magnet having high magnetic flux intensity is used as the magnetic powder for the magnet portion 10. Thus, a ratio of the silicone rubber in the magnet portion 10 can be increased while ensuring a magnetic effect. This allows the magnetic necklace 1 to have enhanced flexibility so as to more desirably come into contact with the user's skin. In cases where the ratio of the silicone rubber is not increased, the magnetic necklace 1 can be reduced in size while ensuring a desired flexibility.

In the above embodiment, each of the lock portion 22 to be provided to the sheath portion 20, and the engagement portion 26 adapted to be releasably locked by the lock portion 22, is formed from a silicone rubber. This allows for fusion between the sheath portion 20 and each of the lock portion 22 and the engagement portion 26, so as to facilitate forming the lock portion 22 and the engagement portion 26 in the sheath portion 20, and suppress separation of the lock portion 22 and the engagement portion 26 from the sheath portion 20.

In the aforementioned production method for the magnetic necklace 1, the magnet-side mixture is extruded while simultaneously extruding the sheath-side mixture onto the outer periphery of the magnet-side mixture, in the extrusion step. This makes it possible to facilitate covering the magnet portion 10 by the sheath portion 10. In the vulcanization step, the extruded magnet-side and sheath-side mixtures are simultaneously vulcanized to achieve fusion between the silicone rubber in the magnet-side mixture and the silicone rubber in the sheath-side mixture so as to facilitate integration between the magnet and sheath portions 10, 20 to be formed from the respective vulcanized mixtures. Particularly, in the vulcanization step, each of the magnet-side and sheath-side mixtures is cured based on the vulcanization of the silicone rubber contained in each of the magnet-side and sheath-side mixtures. That is, the two extruded mixtures are simultaneously vulcanized without vulcanizing each of them under a different condition. This makes it possible to reduce complexity in the production process and simplify production equipment.

An advantageous embodiment of the invention has been shown and described. It is obvious to those skilled in the art that various changes and modifications may be made therein without departing from the spirit and scope thereof as set forth in appended claims. For example, while the above embodiment has been described based on one example where the mixed powder consisting of a powder of rear-earth magnet and a powder of ferrite magnet is used as the magnet powder for the magnet portion 10, the magnet powder to be used in the present invention is not limited to such a mixed powder, but may be any other suitable magnetic powder, such as a samarium-iron-nitrogen based magnet.

Further, while the mixing ratio of the magnetic powder to the silicone rubber in the above embodiment is set at about 7:3, the present invention is not limited to this specific ratio. In view of balance between flexibility and magnetic intensity, the mixing ratio of the magnetic powder to the silicone rubber is preferably set in the range of about 5:5 to 9:1.

The diameter of the magnet portion 10 and the thickness of the sheath portion 20 may be appropriately changed.

In addition to a magnetic necklace, the present invention may be applied to any other magnetic therapeutic device.

The sheath portion may contain another material, such as paint or colorant, according to need.

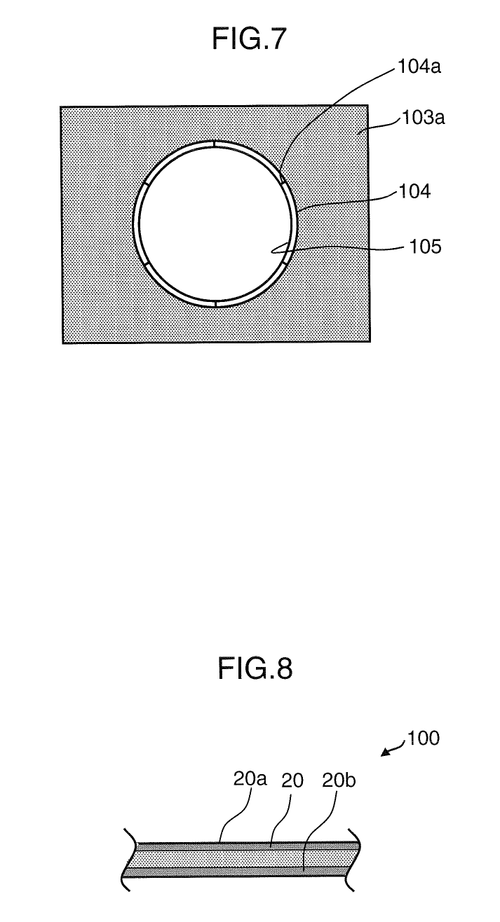

In the above embodiment, the sheath portion 20 is formed from the single type of mixture. Alternatively, the sheath portion 20 may be formed from plural types of mixtures different in color. For example, as shown in FIG. 7, the combining unit in the above embodiment may have a die provided with a plurality of partition plates 104a which divide a space between an inner frame 105 and an outer frame 104 into a plurality of sub-spaces, and the second extruder in the above embodiment may comprise a plurality of second extruders, wherein the mixtures different in color are extruded from the respective sub-spaces divided by the partition plates 104a. In this case, as shown in FIG. 8, a magnet necklace 100 having a sheath portion 20 comprising a plurality of sub-sheath portions 20a, 20b different in color can be provided.

As described above, a magnetic therapeutic device is used as a wearable article including a necklace to exert a blood flow-promoting effect on a body of a user. The magnetic therapeutic device comprises an elongated flexible magnet portion formed from a mixture of a silicone rubber and a magnetic powder to extend in a given direction, and magnetically polarized, and a flexible sheath portion formed from a silicone rubber to cover over an entire outer periphery of the magnet portion.

In the magnetic therapeutic device, the magnet portion is formed from the silicone rubber-and-magnetic powder mixture, and the sheath portion covering over the magnet portion is formed from the silicone rubber. This allows the magnet portion and the sheath portion to be in contact with each other with sufficient conformability, so as to facilitate uniformization of a magnetic flux to be emitted outside from the magnet portion. That is, in the magnetic therapeutic device, a magnetic flux from the magnet portion is uniformly applied to a body of a user, so that a blood flow-promoting effect based on the magnetic flux can be effectively exerted on the user's body. In addition, as mentioned above, the sheath portion in the magnetic therapeutic device is formed from the silicone rubber having physiological stability. Thus, even when the magnetic therapeutic device is in direct contact with a skin of the user, an adverse effect of the sheath portion on the skin can be reliably suppressed.

Preferably, the magnetic therapeutic device may be formed by subjecting the silicone rubber-and-magnetic powder mixture for the magnet portion and the silicone rubber for the sheath portion, to a multicolor extrusion molding process.

This feature makes it possible to facilitate forming the sheath portion in such a manner as to cover over the outer periphery of the magnet portion, while allowing the magnet portion and the sheath portion to more reliably come into contact with each other during the extrusion molding process.

Preferably, in the magnetic therapeutic device, the magnetic powder may be a powder of rare-earth magnet.

According to this feature, a rear-earth magnet having higher magnetic flux intensity than that of a conventional magnetic material for the magnet portion, such as a ferrite magnet, is used as the magnetic powder. Thus, even if a quantity of the magnetic powder for the magnet portion is reduced, a desired magnetic effect of the magnet portion can be maintained. This makes it possible to achieve a reduction in overall size of the device while ensuring the magnetic effect.

In addition, if it is permitted to reduce a quantity of the magnetic powder while ensuring the same level of magnetic effect as described manner, a ratio of the silicone rubber for the magnet portion can be increased while maintaining the magnetic effect and the size of the magnetic therapeutic device at the same levels as those of conventional devices. This makes it possible to provide higher resilience to the magnet portion and achieve enhanced flexibility of the device in its entirety.

Preferably, the magnetic therapeutic device may include a lock portion formed from a silicone rubber at one of opposite ends of the sheath portion, and an engagement portion formed from a silicone rubber at the other end of the sheath portion, and adapted to be releasably locked by the lock portion, wherein the magnetic therapeutic device is adapted, when the engagement portion is locked by the lock portion, to have a ring shape while maintaining the condition where the sheath portion covers over the magnet portion.

This feature allows the magnetic therapeutic device to have enhanced wearability and usability as a wearable article, such as a necklace or a bracelet. Particularly, each of the lock portion and the engagement portion to be provided to the sheath portion is formed from the silicone rubber as with the sheath portion, so as to allow for fusion between the sheath portion and each of the lock portion and the engagement portion. This makes it possible to facilitate forming the lock portion and the engagement portion in the sheath portion, and more reliably suppress undesirable separation, e.g., drop-off, of the lock portion and the engagement portion from the sheath portion.

Further, a method of producing a magnetic therapeutic device comprises: a kneading step of kneading the silicone rubber and magnetic powder for the magnet portion, together with a vulcanizing agent, in a first kneader, and kneading the silicone rubber for the sheath portion, together with a vulcanizing agent, in a second kneader; a charging step of charging a magnet-side mixture of the kneaded silicone rubber, magnetic powder and vulcanizing agent, into a first extruder, and charging a sheath-side mixture of the kneaded silicone rubber and vulcanizing agent, into a second extruder; an extrusion step of extruding the magnet-side mixture from the first extruder, and simultaneously extruding the sheath-side mixture from the second extruder onto an outer periphery of the magnet-side mixture; and a vulcanization step of applying heat to the two extruded mixtures to vulcanize the mixtures.

In the method, each of the magnet-side mixture and the sheath-side mixture can be sufficiently kneaded in the kneading step to suppress a variation in mixed state in each of the magnet portion and the sheath portion. This makes it possible to achieve uniformization of a magnetic effect of the magnetic powder to be exerted on a body of a user. Furthermore, in this method, the magnet-side and sheath-side mixtures are charged into respective ones of the first and second extruders, in the charging step, and the magnet-side mixture is extruded while simultaneously extruding the sheath-side mixture onto the outer periphery of the magnet-side mixture, in the extrusion step. This makes it possible to facilitate covering the magnet-side mixture by the sheath-side mixture. Then, in the vulcanization step, the two extruded mixtures are simultaneously vulcanized. This makes it possible to achieve fusion between the silicone rubber in the magnet-side mixture and the silicone rubber in the sheath-side mixture so as to facilitate integration between the magnet and sheath portions to be formed from the respective vulcanized mixtures. Particularly, in the magnetic therapeutic device, each of the magnet-side mixture and the sheath-side mixture may contain the same type of silicone rubber, so that the two extruded mixtures can be simultaneously vulcanized without vulcanizing each of them under a different condition. This makes it possible to reduce complexity in the production process and simplify production equipment.

The magnetic therapeutic device can uniformly apply a magnetic flux to a body of a user while more reliably suppressing an adverse effect on a skin of the user. The method can effectively produce a magnetic therapeutic device in an efficient manner.

This application is based on Japanese Patent Application No. 2006-339986 filed in Japan Patent Office on Dec. 18, 2006 and Japanese Patent Application No. 2007-014472 filed in Japan Patent Office on Jan. 25, 2007, the contents of which are hereby incorporated by reference.

As this invention may be embodied in several forms without departing from the spirit of essential characteristics thereof, the present embodiment is therefore illustrative and not restrictive, since the scope of the invention is defined by the appended claims rather than by the description preceding them, and all changes that fall within metes and bounds of the claims, or equivalence of such metes and bounds are therefore intended to embraced by the claims.

What is claimed is:

1. A magnetic therapeutic device for use as a wearable article to exert a blood flow-promoting effect on a body of a user, comprising:
an elongated flexible magnet portion formed from a mixture of a silicone rubber and a magnetic powder to extend in a given direction, and magnetically polarized; and
a flexible sheath portion formed from a silicone rubber to cover over an entire outer periphery of said magnet portion.

2. The magnetic therapeutic device as defined in claim 1, which is formed by subjecting said silicone rubber-and-magnetic powder mixture for said magnet portion and said silicone rubber for said sheath portion, to a multicolor extrusion molding process.

3. The magnetic therapeutic device as defined in claim 1, wherein said magnetic powder is a powder of rare-earth magnet.

4. The magnetic therapeutic device as defined in claim 1, which includes:
a lock portion formed from a silicone rubber at one of opposite ends of said sheath portion; and
an engagement portion formed from a silicone rubber at the other end of said sheath portion, and adapted to be releasably locked by said lock portion,
wherein said magnetic therapeutic device is adapted, when said engagement portion is locked by said lock portion, to have a ring shape while maintaining the condition where said sheath portion covers over said magnet portion.

5. A method of producing the magnetic therapeutic device as defined in claim 2, comprising:
a kneading step of kneading the silicone rubber and magnetic powder for said magnet portion, together with a vulcanizing agent, in a first kneader, and kneading the silicone rubber for said sheath portion, together with a vulcanizing agent, in a second kneader;
a charging step of charging a magnet-side mixture of said kneaded silicone rubber, magnetic powder and vulcanizing agent, into a first extruder, and charging a sheath-side mixture of said kneaded silicone rubber and vulcanizing agent, into a second extruder;
an extrusion step of extruding said magnet-side mixture from said first extruder, and simultaneously extruding said sheath-side mixture from said second extruder onto an outer periphery of said magnet-side mixture; and
a vulcanization step of applying heat to said two extruded mixtures to vulcanize said mixtures.
